SEQUENCE LISTING (3 sequences)

SEQ ID NO: 1 - DNA, 1362 bp, Homo sapiens
  orphan G-protein-coupled receptor (GPR) 39
atggcttcacccagcctcccgggcagtgactgctcccaaatcattgatcacagtcatgtccccgagtttgaggtggccacctggatcaaaatcacccttattctggtgtacctgatcatcttcgtgatgggccttctggggaacagcgccaccattcgggtcacccaggtgctgcagaagaaaggatacttgcagaaggaggtgacagaccacatggtgagtttggcttgctcggacatcttggtgttcctcatcggcatgcccatggagttctacagcatcatctggaatcccctgaccacgtccagctacaccctgtcctgcaagctgcacactttcctcttcgaggcctgcagctacgctacgctgctgcacgtgctgacactcagctttgagcgctacatcgccatctgtcaccccttcaggtacaaggctgtgtcgggaccttgccaggtgaagctgctgattggcttcgtctgggtcacctccgccctggtggcactgcccttgctgtttgccatgggtactgagtaccccctggtgaacgtgcccagccaccggggtctcacttgcaaccgctccagcacccgccaccacgagcagcccgagacctccaatatgtccatctgtaccaacctctccagccgctggaccgtgttccagtccagcatcttcggcgccttcgtggtctacctcgtggtcctgctctccgtagccttcatgtgctggaacatgatgcaggtgctcatgaaaagccagaagggctcgctggccgggggcacgcggcctccgcagctgaggaagtccgagagcgaagagagcaggaccgccaggaggcagaccatcatcttcctgaggctgattgttgtgacattggccgtatgctggatgcccaaccagattcggaggatcatggctgcggccaaacccaagcacgactggacgaggtcctacttccgggcgtacatgatcctcctccccttctcggagacgtttttctacctcagctcggtcatcaacccgctcctgtacacggtgtcctcgcagcagtttcggcgggtgttcgtgcaggtgctgtgctgccgcctgtcgctgcagcacgccaaccacgagaagcgcctgcgcgtacatgcgcactccaccaccgacagcgcccgctttgtgcagcgcccgttgctcttcgcgtcccggcgccagtcctctgcaaggagaactgagaagattttcttaagcacttttcagagcgaggccgagccccagtctaagtcccagtcattgagtctcgagtcactagagcccaactcaggcgcgaaaccagccaattctgctgcagagaatggttttcaggagcatgaagtttga

SEQ ID NO: 2 - protein, 453 aa, Homo sapiens
  orphan G-protein-coupled receptor (GPR) 39
MASPSLPGSDCSQIIDHSHVPEFEVATWIKITLILVYLIIFVMGLLGNSATIRVTQVLQKKGYLQKEVTDHMVSLACSDILVFLIGMPMEFYSIIWNPLTTSSYTLSCKLHTFLFEACSYATLLHVLTLSFERYIAICHPFRYKAVSGPCQVKLLIGFVWVTSALVALPLLFAMGTEYPLVNVPSHRGLTCNRSSTRHHEQPETSNMSICTNLSSRWTVFQSSIFGAFVVYLVVLLSVAFMCWNMMQVLMKSQKGSLAGGTRPPQLRKSESEESRTARRQTIIFLRLIVVTLAVCWMPNQIRRIMAAAKPKHDWTRSYFRAYMILLPFSETFFYLSSVINPLLYTVSSQQFRRVFVQVLCCRLSLQHANHEKRLRVHAHSTTDSARFVQRPLLFASRRQSSARRTEKIFLSTFQSEAEPQSKSQSLSLESLEPNSGAKPANSAAENGFQEHEV

SEQ ID NO: 3 - protein, 200 aa, Artificial Sequence
  Description of Artificial Sequencepoly Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

(19) United States
(12) Patent Application Publication
(10) Pub. No.: US 2004/0071708 A1
(43) Pub. Date: Apr. 15, 2004

(54) GPR 39 modulators that control cancerous cell growth

(75) Inventors: Gisela Claassen, San Diego, CA (US); Henry Li, Carlsbad, CA (US); Jack Barber, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Immusol, Inc., San Diego, CA

(21) Appl. No.: 10/255,775

(22) Filed: Sep. 26, 2002

(51) Int. Cl. G01N 33/574; A61K 48/00; A61K 39/395

(52) U.S. Cl. 424/155.1; 514/44; 435/7.23

(57) ABSTRACT

The present invention discloses compositions, systems and methods for identifying anti-cancer agents using doth in vitro and in vivo techniques. Embodiments include the screening of combinatorial libraries of peptides, antibodies, and small organic molecules as well as siRNAs, ribozymes and antisense nucleotides directed against nucleic acids encoding the GPR 39 protein.

### CROSS-REFERENCE TO RELATED APPLICATIONS

[Not Applicable ]

### STATEMENT AS TO RIGHTS TO INVENTIONS MADE UNDER FEDERALLY SPONSORED RESEARCH AND DEVELOPMENT

[Not Applicable ]

### FIELD OF THE INVENTION

The present invention discloses compositions, systems and methods for identifying anti-cancer agents using both in vitro and in vivo techniques. Embodiments include the screening of combinatorial libraries of peptides, antibodies, and small organic molecules as well as siRNAs, ribozymes and antisense nucleotides directed against nucleic acids encoding the GPR 39 protein.

### BACKGROUND OF THE INVENTION

The actions of many extracellular signals are mediated by the interaction of guanine nucleotide-binding regulatory proteins (G proteins) and G-protein coupled receptors (GPCRs). Individual GPCRs activate particular signal transduction pathways through binding to G proteins, which in turn transduce a signal to the cell to elicit a response from the cell. GPCRs are known to respond to numerous extracellular signals, including neurotransmitters, drugs, hormones, odorants and light. The family of GPCRs has been estimated to include several thousands members, fully more than 1.5% of all the proteins encoded in the human genome. The GPCR family members play key roles in regulation of biological phenomena involving virtually every cell in the body. The sequencing of the human genome has led to identification of numerous GPCRs; although a significant portion of these identified receptors are without known ligands. These latter GPCRs, known as “orphan receptors”, have unknown physiological roles, but considering the importance of GPCRs in cellular signal transduction, are likely candidates as targets for pharmaceutical compounds.

Indeed, many available therapeutic drugs in use today target GPCRs that mediate vital physiological responses, including vasodilation, heart rate, bronchodilation, endocrine secretion, and gut peristalsis. See, eg., Lefkowitz et al., Ann. Rev. Biochem. 52:159 (1983); Gilman, A. G. (1987) Annu. Rev. Biochem 56:615-649; Hamm, H. E. (1998) JBC 273:669-672; Ji, T. H. (1998) JBC 273:17229-17302. Kanakin, T. (1996) Pharmacological review, 48:413-463; Gudermann T. and Schultz, G. (1997), Annu. Rev. Neurosci., 20:399-427. In fact, it has been estimated that more than 50% of the drugs in use clinically in humans at the present time are directed at GPCRs, including the adrenergic receptors (ARs). For example ligands to beta ARs are used in the treatment of anaphylaxis, shock, hypertension, hypotension, asthma and other conditions. As the ligand(s) specifically recognizing orphan GPCRs are not known, new methods for rapidly screening compounds capable of specifically recognizing orphan GPCRs are needed before new treatment regimes can be devised. This is particularly true for orphan GPCRs that have been implicated in disease states, where modulators of receptor activity of potential therapeutic value.

### SUMMARY OF THE INVENTION

The invention provides methods, systems and compositions for identifying chemotherapeutic agents for the treatment of certain cancers sensitive to the modulation of the G-protein coupled receptor, GPR 39. In one embodiment the invention provides a method of identifying anticancer agents that modulate GPR 39 protein. By GPR 39 protein is meant any protein that has at least 60%, more preferably 75%, even more preferably 90% and most preferably 95%, 98% or 99% homology to SEQ ID NO: 2. The method comprises contacting GPR 39-specific binding agents to cancer cells, followed by detecting anticancer activity to identify an anticancer agent. The GPR 39-specific binding agents contacted to the cancer cells can be part of a single composition, or tested as a series of separate compositions. In some aspects, the cancer cells contacted comprise a BRCA-1 phenotype.

Another method of the invention inhibits cancer characteristics in cancer cells by downmodulating GPR 39 protein activity to a level sufficient to inhibit the cancer characteristics of the cancer cells. The method can use intrabodies, antisense molecules ribozymes, siRNAs, or antibodies that antagonize GPR 39 activity or expression.

A system for identifying anticancer agents that modulate GPR 39 protein is also provided. This system comprises a container containing GPR 39-specific binding agents and a container housing cancer cells that express GPR 39 protein. As in the method described above, the GPR 39 protein is any protein that has at least 60%, more preferably 75%, even more preferably 90% and most preferably 95%, 98% or 99% homology to SEQ ID NO: 2. The system is useful in identifying both BRCA-1 sensitive and BRCA-1 insensitive cancer cells originating from various tissues including, breast, ovarian, prostate, brain, and lung cancer cells.

A number of different binding agents can be used in practicing the system. These include nucleic acids, antibodies and various peptides, including peptides that bind to the transmembrane portion of the GPR 39 protein.

One embodiment of the invention includes a recombinant expression cassette comprising a non-native promoter operably linked to a gene encoding human GPR 39 protein. The encoded GPR 39 protein has at least 60%, more preferably 75%, even more preferably 90% and most preferably 95%, 98% or 99% homology to SEQ ID NO: 2.

Another embodiment of the invention is an antibody specifically recognizing a peptide having a sequence of at least five amino acids found in a GPR 39 protein having at least 60%, more preferably 75%, even more preferably 90% and most preferably 95%, 98% or 99% homology to SEQ ID NO: 2. In some aspects, the antibody will down-regulate GPR 39 activity when it binds the protein. In other aspects the antibody will up regulate the protein. The antibody can be a single chain antibody, or an intrabody as well as any of the immunoglobulin fragments defined as antibodies herein. Preferably, binding of the antibody to GPR 39 protein will inhibit the cancer characteristics of the cell expressing the bound receptor.

Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Anticancer activity” refers to an ability prevent, retard or reverse a cancerous phenotype.

“Anticancer agent” refers to an material or composition with anticancer activity.

An “antisense molecule” refers to a polynucleotide that is complementary to a target sequence of choice and capable of specifically hybridizing with the target molecules. The term antisense includes a “ribozyme,” which is a catalytic RNA molecule that cleaves a target RNA through ribonuclease activity. Antisense nucleic acids hybridize to a target polynucleotide and interfere with the transcription, processing, translation or other activity of the target polynucleotide. An antisense nucleic acid can inhibit DNA replication or DNA transcription by, for example, interfering with the attachment of DNA or RNA polymerase to the promoter by binding to a transcriptional initiation site or a template. It can interfere with processing of mRNA, poly(A) addition to mRNA or translation of mRNA by, for example, binding to regions of the RNA transcript such as the ribosome binding site. It can promote inhibitory mechanisms of the cells, such as promoting RNA degradation via RNase action. The inhibitory polynucleotide can bind to the major groove of the duplex DNA to form a triple helical or “triplex” structure. Methods of inhibition using antisense polynucleotides therefore encompass a number of different approaches to altering expression of specific genes that operate by different mechanisms (see, e.g., Helene & Toulme, Biochim. Biophys. Acta., 1049:99-125 (1990)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—CH1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “BRCA-1 sensitive phenotype” describes a cancer cell or cell line that has a reduced level of BRCA-1 when compared to normal tissue samples from the same tissue source as the cancer cell or cell line was derived. “BRCA-1 sensitive phenotype” also includes cancer cells that show a remission of cancer characteristics in response to up-regulation of cellular BRCA-1 expression.

“Cancer characteristics” refers to phenotypic and morphologic cellular changes typically found in cancer cells. Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia.

Pre-malignant abnormal cell growth is exemplified by hyperplasia, metaplasia, or most particularly, dysplasia (for review of such abnormal growth conditions, see Robbins and Angell, 1976, Basic Pathology, 2d Ed., W. B. Saunders Co., Philadelphia, pp. 68-79.) Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

The neoplastic lesion may evolve clonally and develop an increasing capacity for invasion, growth, metastasis, and heterogeneity, especially under conditions in which the neoplastic cells escape the host's immune surveillance (Roitt, I., Brostoff, J and Kale, D., 1993, Immunology, 3rd ed., Mosby, St. Louis, pps. 17.1-17.12).

“cancer cells” refers to cells displaying cancer characteristics. A cancer cell can occur in and can be obtained from a solid tumor such as a sarcoma, carcinoma, melanoma, lymphoma or glioma or a more diffuse cancer such as a leukemia. Cancer cells can be obtained from a subject having a cancer, from a donor subject having a cancer that is the same or substantially similar to the cancer n the subject to be treated or from a cancer cell repository.

Cancer cells are frequently defined by the normal “source cell” from which the cancerous cell is derived, for example, “breast cancer cells” originate from breast tissue, “lung cancer cells” from lung tissue, “prostate cancer cells” from cells of the prostate and “ovarian cancer cells” from the ovary.

“GPR 39 protein” refers to an orphan G protein-coupled receptor with a nucleic acid sequence having at least 60%, preferably at least 75% more preferably 90%, still more preferably 95%, preferably at least 98% and most preferably 99% homology with the amino acid sequence of SEQ ID: 2. A “GPR 39 protein coding sequence” refers to any nucleic acid encoding a GPR 39 protein, as defined herein.

“GPR 39-specific binding agents” refers t o compounds and compositions that preferentially bind to a GPR 39 receptor. To be considered preferential binding, the compound or composition will bind to a GPR 39 receptor with an affinity at least 2 times, preferably 4 times, more preferably 10 times, most preferably at least 20 times greater than the affinity of the compound or composition to another receptor type.

The terms “sequence similarity”, “sequence identity”, or “percent identity,” in the context of two or more nucleic acids or polypeptide sequences, refer to two or more sequences or subsequences that are, when optimally aligned with appropriate nucleotide insertions or deletions, the same or have a specified percentage of amino acid residues or nucleotides that are the same (i.e., 50% identity, 65%, 70%, 75%, 80%, preferably 85%, 90%, 91%, 92%, 93%, 94%, 95%, 96%, 97%, 98%, 99% or higher identity to an amino acid sequence such as SEQ ID NO: 2, or a nucleotide sequence such as SEQ ID NO: 1), when compared and aligned for maximum correspondence over a comparison window, or designated region as measured using one of the following sequence comparison algorithms or by manual alignment and visual inspection. This definition also refers to the compliment of a test sequence. Preferably, the identity exists over a region that is at least about 25 amino acids or nucleotides in length, or more preferably over a region that is 50-100 amino acids or nucleotides in length. These relationships hold, notwithstanding evolutionary origin (Reeck et al., Cell, 50:667 (1987)). When the sequence identity of a pair of polynucleotides or polypeptides is greater or equal to 65%, the sequences are said to be “substantially identical.”

Alternatively, substantial identity will exist when a nucleic acid will hybridize under selective hybridization conditions, to a strand or its complement. Typically, selective hybridization will occur when there is at least about 55% homology over a stretch of at least about 14 nucleotides, more typically at least about 65%, preferably at least about 75%, and more preferably at least about 90%. See, Kanehisa, Nuc. Acids Res., 12:203-213 (1984), which is incorporated herein by reference. The length of homology comparison, as described, may be over longer stretches, and in certain embodiments will be over a stretch of at least about 17 nucleotides, generally at least about 20 nucleotides, ordinarily at least about 24 nucleotides, usually at least about 28 nucleotides, typically at least about 32 nucleotides, more typically at least about 40 nucleotides, preferably at least about 50 nucleotides, and more preferably at least about 75 to 100 or more nucleotides.

Amino acid sequence homology, or sequence identity, is determined by optimizing residue matches, if necessary, by introducing gaps as required. This changes when considering conservative substitutions as matches. Conservative substitutions typically include substitutions within the following groups: [glycine, alanine]; [valine, isoleucine, leucine]; [aspartic acid, glutamic acid]; [asparagine, glutamine]; [serine, threonine]; [lysine, arginine]; and [phenylalanine, tyrosine]. Homologous amino acid sequences are intended to include natural allelic and interspecies variations in each respective receptor sequence. Typical homologous proteins or peptides will have from 25-100% homology (if gaps can be introduced), to 50-100% homology (if conservative substitutions are included). Homology measures will be at least about 50%, generally at least 56%, more generally at least 62%, often at least 67%, more often at least 72%, typically at least 77%, more typically at least 82%, usually at least 86%, more usually at least 90%, preferably at least 93%, and more preferably at least 96%, and in particularly preferred embodiments, at least 98% or more.

In relation to proteins, the term “homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell, 50:667 (1987)). The present invention naturally contemplates homologues of the GPR 39 protein, and polynucleotides encoding the same, as falling within the scope of the invention.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Intrabody” refers to a class of neutralizing molecules with applications in gene therapy (vonMehren M, Weiner L M. (1996) Current Opinion in Oncology. 8:493-498, Marasco Wash. (1997) Gene Therapy. 4:11-15, Rondon I J, Marasco Wash. (1997) Annual Review of Microbiology. 51:257-283).

The term “modulate” refers to an ability to increase or decrease a detectable characteristic.

“siRNA” refers to a ds RNA that is preferably between 16 and 25, more preferably 17 and 23 and most preferably between 18 and 21 base pairs long, each strand of which has a 3′ overhang of 2 or more nucleotides. Functionally, the characteristic distinguishing an siRNA over other forms of dsRNA is that the siRNA comprises a sequence capable of specifically inhibiting genetic expression of a gene or closely related family of genes by a process termed RNA interference.

“Non-native promoter” refers to any promoter element operably linked to a coding sequence by recombinant methods. Non-native promoters include mutagenized native reporters, when mutagenesis alters the rate or control of transcriptional events.

“Operably linked” refers to a linkage of polynucleotide elements in a functional relationship. With regard to the present invention, the term “operably linked” refers to a functional linkage between a nucleic acid expression control sequence (such as a promoter, or an array of transcription factor binding sites) and a second nucleic acid sequence, wherein the expression control sequence directs transcription of the nucleic acid corresponding to the second sequence. Thus, a nucleic acid is “operably linked” when it is placed into a functional relationship with another nucleic acid sequence

“Peptide” refers to any of various natural or synthetic compounds containing two or more amino acids linked by the carboxyl group of one amino acid to the amino group of another.

“Recombinant expression cassette” refers to a DNA sequence capable of directing expression of a nucleic acid in cells. A “DNA expression cassette” comprises a promoter, operably linked to a nucleic acid of interest, which is further operably linked to a termination region.

“Transmembrane portion of the GPR 39 protein” refers to seven distinctly hydrophobic regions of the peptidyl chain that are between twenty to thirty amino acids in length and believed to span the cellular membrane.

### DETAILED DESCRIPTION

Before the present modified proteins and methods are described, it is to be understood that this invention is not limited to particular constructs and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

I. Introduction

GPR 39 has been identified as a G protein coupled receptor based on a characteristic transmembrane domain motif found in the primary structure of the protein. Sequence analysis indicates that GPR 39 belongs to the GHS-R/NT-Rs (neurotensin receptor) family. Although GPR 39 is widely expressed in several tissues and implicated in growth control and cancer, no ligand has been reported for this receptor.

The present invention is based on the finding that down-regulation of GPR 39 in cancer cells leads to a remission of cancer phenotypic characteristics to those of a normal cell. Moreover, in certain forms of breast cancer where BRCA-1 expression is depressed, downregulation of GPR 39 results in a remission of the cancerous phenotype. Accordingly, the present invention provides methods, systems and antibodies for the identification of compounds that specifically recognize and bind GPR 39. In one embodiment, the present invention provides compositions and assays that identify compounds modulating GPR 39 by specifically binding to the GPR 39 protein and leading to a remission of cancer characteristics when applied to cancer cells.

Furthermore, GPCR activity is generally modulated via a recycling of the receptor, a mechanism common to all known GPCR's. This common method of modulating receptor activity suggests that agents effecting expression of the receptor or cellular recycling of the receptor protein may also prove effective sites of drug interaction. Accordingly, another embodiment of the present invention provides methods for identifying and constructing ribozymes, siRNAs, antisense nucleotides and intrabodies directed against GPR 39 and designed to down-regulate GPR 39 expression causing remission of cancer characteristics when applied to cancer cells, independent of receptor binding.

Other embodiments of the present invention are systems comprised of components useful in practicing the methods of the invention. Typical components of these systems include cancer cells expressing native or recombinant forms of the GPR 39 protein, and specific GPR 39 modulators, including combinatorial libraries of potential GPR 39 modulators.

II. Sources of GPR 39

A gene encoding GPR 39 protein, whether genomic DNA or cDNA, can be isolated from any source, particularly from a human cDNA or genomic library. The coding sequence for one GPR 39 protein is provided as SEQ ID: 1, and the sequence of the corresponding protein is provided as SEQ ID: 2. Methods for obtaining GPR 39 gene(s) are well known in the art. (see, e.g., Sambrook et al., (1989) Molecular Cloning A Laboratory Manual (2^{nd} ed.) Vol. 1-3, Cold Spring Harbor Laboratory, Cold Spring Harbor Press, N.Y., (Sambrook)). The DNA preferably is obtained from a cDNA library prepared from tissues with high level expression of the protein (e.g., a brain or ovarian cell library, since these are the cells that evidence highest levels of expression of GPR 39), by chemical synthesis, by cDNA cloning, or by the cloning of genomic DNA, or fragments thereof, purified from the desired cell (See, for example, Sambrook et al, 1989, supra; Glover, D. M. (ed.), 1985, DNA Cloning: A Practical Approach, MRL Press, Ltd., Oxford, U. K. Vol. I, II).

A. Cloning Techniques

Whatever the source, the gene should be molecularly cloned into a suitable vector for propagation and amplification. Identification of the specific DNA fragment containing the desired GPR 39 gene may be accomplished in a number of ways. For example, a portion of a GPR 39 gene can be purified and labeled to prepare a labeled probe, and the generated DNA may be screened by nucleic acid hybridization to the labeled probe (Benton and Davis, Science 196:180, 1977; Grunstein and Hogness, Proc. Natl. Acad. Sci. U.S.A. 72:3961, 1975). Those DNA fragments with substantial homology to the probe, such as an allelic variants, will hybridize.

Standard techniques for construction of the expression cassettes and vectors for propagation and amplification of coding sequences are well known to those of ordinary skill in the art (Sambrook, J., Fritsch, E. F., and Maniatus, T., Molecular Cloning, A Laboratory Manual 2nd ed. (1989); Gelvin, S. B., Schilperoort, R. A., Varma, D. P. S., eds. Plant Molecular Biology Manual (1990)). A variety of strategies are available for ligating fragments of DNA, the choice depending on the nature of the termini of the DNA fragments.

In preparing the expression cassette, the various DNA sequences may normally be inserted or substituted into a bacterial plasmid. Any convenient plasmid may be employed, which will be characterized by having a bacterial replication system, a marker which allows for selection in the bacterium and generally one or more unique, conveniently located restriction sites.

1. Genetic modifications to GPR 39

Several of the techniques described herein comprise modified GPR 39, particularly GPR 39 isoforms that are constitutively active. One of skill in the art will recognize many ways of generating alterations in a given nucleic acid sequence. Such well-known methods include site-specific mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Product information from manufacturers of biological reagents and experimental equipment also provide information useful in known biological methods. Such manufacturers include the SIGMA Chemical Company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.), as well as many other commercial sources known to one of skill. Using these techniques, it is possible to insert or delete, at will, a polynucleotide of any length into a DNA expression cassette described herein.

a) Site-Directed Mutagenesis

Site-directed mutagenesis techniques are described in Ling et al., “Approaches to DNA mutagenesis: an overview”, Anal Biochem., 254(2):157-178 (1997); Dale et al., “In vitro mutagenesis”, Ann. Rev. Genet., 19:423-462 (1996); Botstein & Shortle, “Strategies and applications of in vitro mutagenesis”, Science, 229:1193-1201 (1985); Carter, “Site-directed mutagenesis”, Biochem. J., 237:1-7 (1986); and Kunkel, “The efficiency of oligonucleotide directed mutagenesis” in Nucleic Acids & Molecular Biology (Eckstein, F. and Lilley, D. M. J. eds., Springer Verlag, Berlin) (1987)); mutagenesis using uracil containing templates (Kunkel, “Rapid and efficient site-specific mutagenesis without phenotypic selection”, Proc. Natl. Acad. Sci. USA, 82:488-492 (1985); Kunkel et al., “Rapid and efficient site-specific mutagenesis without phenotypic selection”, Methods in Enzymol., 154:367-382 (1987); and Bass et al. (1988); oligonucleotide-directed mutagenesis (Methods in Enzymol., 100:468-500 (1983); Methods in Enzymol., 154:329-350 (1987); Zoller & Smith, “Oligonucleotide-directed mutagenesis using M13-derived vectors: an efficient and general procedure for the production of point mutations in any DNA fragment”, Nucleic Acids Res., 10:6487-6500 (1982); Zoller & Smith “Oligonucleotide-directed mutagenesis of DNA fragments cloned into M13 vectors”, Methods in Enzymol., 100:468-500 (1983); and Zoller & Smith, “Oligonucleotide-directed mutagenesis: a simple method using two oligonucleotide primers and a single-stranded DNA template”, Methods in Enzymol., 154:329-350 (1987)); Taylor et al. (1985) “The rapid generation of oligonucleotide-directed mutations at high frequency using phosphorothioate-modified DNA”, Nucl. Acids Res., 13:8765-8787 (1985); Nakamaye & Eckstein, “Inhibition of restriction endonuclease Nci I cleavage by phosphorothioate groups and its application to oligonucleotide-directed mutagenesis”, Nucl. Acids Res., 14:9679-9698 (1986); Sayers et al., “Y-T Exonucleases in phosphorothioate-based oligonucleotide-directed mutagenesis”, Nucl. Acids Res., 16:791-802 (1988); and Sayers et al. (1988); mutagenesis using gapped duplex DNA (Kramer et al., “The gapped duplex DNA approach to oligonucleotide-directed mutation construction”, Nucl. Acids Res., 12:9441-9456 (1984); Kramer & Fritz, “Oligonucleotide-directed construction of mutations via gapped duplex DNA”, Methods in Enzymol., 154:350-367 (1987); Kramer et al., “Improved enzymatic in vitro reactions in the gapped duplex DNA approach to oligonucleotide-directed construction of mutations”, Nucl. Acids Res., 16:7207 (1988); and Fritz et al., “Oligonucleotide-directed construction of mutations: a gapped duplex DNA procedure without enzymatic reactions in vitro”, Nucl. Acids Res., 16:6987-6999 (1988)).

Other techniques for altering DNA sequences include; Wells et al., “Cassette mutagenesis: an efficient method for generation of multiple mutations at defined sites”, Gene, 34:315-323 (1985); and Grundstrom et al., “Oligonucleotide-directed mutagenesis by microscale 'shot-gun gene synthesis”, Nucl. Acids Res., 13:3305-3316 (1985)), double-strand break repair (Mandecki, “Oligonucleotide-directed double-strand break repair in plasmids of Escherichia coli: a method for site-specific mutagenesis”, Proc. Natl. Acad. Sci. USA, 83:7177-7181 (1986); and Arnold, “Protein engineering for unusual environments”, Current Opinion in Biotechnology, 4:450-455 (1993)). Additional details on many of the above methods can be found in Methods in Enzymology Volume 154, which also describes useful controls for trouble-shooting problems with various mutagenesis methods.

The sequence of the isolated and synthetic oligonucleotides can be verified after cloning using, e.g., the chain termination method for sequencing double-stranded templates of Wallace et al., Gene, 16:21-26 (1981).

b) PCR Amplification

Polymerase chain reaction, or other in vitro amplification methods, may also be useful, for example, in cloning nucleic acid sequences encoding proteins to be expressed; in making nucleic acids to use as probes for detecting the presence of GPR 39 encoding mRNA in physiological samples; for nucleic acid sequencing, or other purposes (see U.S. Pat. Nos. 4,683,195 and 4,683,202; PCR Protocols: A Guide to Methods and Applications (Innis et al., eds, 1990)). Such methods can be used to PCR amplify GPR 39 nucleic acid sequences directly from mRNA, or from either genomic or cDNA libraries. Degenerate oligonucleotides can be designed to amplify GPR 39 homologues using the sequences provided herein (e.g., SEQ ID NO: 4 to 11). Restriction endonuclease sites can be incorporated into the primers. Genes amplified by the PCR reaction can be purified from agarose gels and cloned into an appropriate vector.

PCR techniques include 5′ and/or 3′ RACE techniques, both being capable of generating a full-length cDNA sequence from a suitable cDNA library (Frohman, et al., Proc. Natl. Acad. Sci. USA, 85:8998-9002 (1988)). The strategy involves using specific oligonucleotide primers for PCR amplification of GPR 39 cDNA. These specific primers are designed through identification of nucleotide sequences either in the cDNA itself, and/or the vector comprising the cDNA.

2. cDNA Libraries

Preparation of cDNA libraries can be performed by standard techniques well known in the art. Well known cDNA library construction techniques can be found for example, in Sambrook et al., 1989, Molecular Cloning: A Laboratory Manual; Cold Spring Harbor Laboratory, Cold Spring Harbor, N.Y.

To make a cDNA library, one should choose a source that is rich in GPR 39 mRNA, e.g., brain or ovarian tissue, or cell lines derived therefrom. The mRNA is then made into CDNA using reverse transcriptase, ligated into a recombinant vector, and transfected into a recombinant host for propagation, screening and cloning. Methods for making and screening cDNA libraries are well known (see, e.g., Gubler & Hoffman, Gene, 25:263-269 (1983); Sambrook et al., supra; Ausubel et al., supra).

A GPR 39-containing cDNA library constructed in a bacteriophage or plasmid shuttle vector can be screened, for example, with a labeled oligonucleotide probe designed from the nucleic acid sequence of SEQ ID NO: 1. The oligonucleotide probe design can be a partial cDNA encoding GPR 39, obtained by specific PCR amplification of GPR 39 DNA fragments using degenerate oligonucleotide primers based on the amino acid sequence determined from N-terminal amino acid sequencing of GPR 39, such as SEQ ID NO: 2. Alternatively PCR amplification techniques, such as those discussed in detail below, can also be used to isolate the GPR 39-encoding cDNA.

It will be readily apparent to those skilled in the art that other types of libraries, as well as libraries constructed from other cell types or species types, may be useful for isolating an GPR 39-encoding DNA or a homologue of an GPR 39-encoding DNA. Other types of libraries include, but are not limited to, cDNA and genomic libraries derived from cells or cell lines other than human cell lines, such as monkey, mice, hamster, rabbit or any other such host which may contain GPR 39-encoding DNA.

4. Genomic Libraries

For a genomic library, the DNA is extracted from the tissue and either mechanically sheared or enzymatically digested to yield fragments of about 12-20 Kb. The fragments are then separated by gradient centrifugation from undesired sizes and are constructed in bacteriophage λ vectors. These vectors and phage are packaged in vitro. Recombinant phage are analyzed by plaque hybridization as described in Benton & Davis, Science, 196:180-182 (1977). Colony hybridization is carried out as generally described in Grunstein et al., Proc. Natl. Acad. Sci. USA., 72:3961-3965 (1975). See also, Gussow, D. and Clackson, T., Nucl. Acids Res., 17:4000 (1989).

3. Chemical Synthesis of Oligonucleotides

Chemical synthesis of linear oligonucleotides is well known in the art and can be achieved by solution or solid phase techniques. Moreover, linear oligonucleotides of defined sequence can be purchased commercially or can be made by any of several different synthetic procedures including the phosphoramidite, phosphite triester, H-phosphonate and phosphotriester methods, typically by automated synthesis methods. The synthesis method selected can depend on the length of the desired oligonucleotide and such choice is within the skill of the ordinary artisan. For example, the phosphoramidite and phosphite triester method produce oligonucleotides having 175 or more nucleotides while the H-phosphonate method works well for oligonucleotides of less than 100 nucleotides. Oligonucleotides are typically synthesized chemically according to the solid phase phosphoramidite triester method described by Beaucage and Caruthers (1981), Tetrahedron Letts., 22(20):1859-1862, e.g., using an automated synthesizer, as described in Needham-VanDevanter et al. (1984) Nucleic Acids Res., 12:6159-6168. Oligonucleotides can also be custom made and ordered from a variety of commercial sources known to persons of skill in the art. Purification of oligonucleotides, where necessary, is typically performed by either native acrylamide gel electrophoresis or by anion-exchange HPLC as described in Pearson and Regnier (1983) J. Chrom. 255:137-149.

Synthetic linear oligonucleotides may be purified by polyacrylamide gel electrophoresis, or by any of a number of chromatographic methods, including gel chromatography and high pressure liquid chromatography. The sequence of the synthetic oligonucleotides can be verified using the chemical degradation method of Maxam and Gilbert (1980) in Grossman and Moldave (eds.) Academic Press, New York, Methods in Enzymology 65:499-560. If modified bases are incorporated into the oligonucleotide, and particularly if modified phosphodiester linkages are used, then the synthetic procedures are altered as needed according to known procedures. In this regard, Uhlmann, et al. (1990, Chemical Reviews 90:543-584) provide references and outline procedures for making oligonucleotides with modified bases and modified phosphodiester linkages. Sequences of short oligonucleotides can also be analyzed by laser desorption mass spectroscopy or by fast atom bombardment (McNeal, et al., 1982, J. Am. Chem. Soc. 104:976; Viari, et al., 1987, Biomed. Enciron. Mass Spectrom. 14:83; Grotjahn et al., 1982, Nuc. Acid Res. 10:4671). Analogous sequencing methods are available for RNA oligonucleotides.

Chemical synthesis of oligonucleotides encoding dsRNA's can also be performed using nucleotide analogs. Use of analogs frequently confers desirable properties to the oligonucleotide, such as resistance to nucleases, or ease of entry into cells during transformation. Preferred nucleotide analogs are unmodified G, A, T, C and U nucleotides; pyrimidine analogs with lower alkyl, alkynyl or alkenyl groups in the 5 position of the base and purine analogs with similar groups in the 7 or 8 position of the base. Other preferred nucleotide analogs are 5-methylcytosine, 5-methyluracil, diaminopurine, and nucleotides with a 2′-O-methylribose moiety in place of ribose or deoxyribose. As used herein lower alkyl, lower alkynyl and lower alkenyl contain from 1 to 6 carbon atoms and can be straight chain or branched. These groups include methyl, ethyl, propyl, isopropyl, butyl, isobutyl, tertiary butyl, amyl, hexyl and the like. A preferred alkyl group is methyl.

B. Expression Cassettes for Prokaryotes and Eukaryotes

To obtain high level expression of a cloned gene or nucleic acid, such as those cDNAs encoding GPR 39, one typically subclones GPR 39 into an expression cassette that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the GPR 39 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one embodiment, the eukaryotic expression cassette is an adenoviral cassette, an adeno-associated cassette, or a retroviral cassette.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression cassette typically contains a transcription unit or expression cassette that contains all the additional elements required for the expression of the GPR 39 encoding nucleic acid in host cells. A typical expression cassette thus contains a promoter operably linked to the nucleic acid sequence encoding GPR 39 and signals required for efficient polyadenylation of the transcript, ribosome binding sites, and translation termination. The nucleic acid sequence encoding GPR 39 may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression cassette used to transport the genetic information into the cell is not particularly critical. Any of the conventional cassettes used for expression in eukaryotic or prokaryotic cells may be used. Standard bacterial expression cassettes include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression cassettes containing regulatory elements from eukaryotic viruses are typically used in eukaryotic expression cassettes, e.g., SV40 cassettes, papilloma virus cassettes, and cassettes derived from Epstein-Barr virus. Other exemplary eukaryotic cassettes include pMSG, pAV009/A⁺, pMTO10/A⁺, pMAMneo-5, baculovirus pDSVE, and any other cassette allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus cassette in insect cells, with a GPR 39 encoding sequence under the direction of the polyhedrin promoter or other strong baculovirus promoters.

The elements that are typically included in expression cassettes also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of GPR 39 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma cassettes, viral cassettes and any of the other well known methods for introducing cloned genomic DNA, CDNA, synthetic DNA or other foreign genetic material into a host cell (see, e.g., Sambrook et al., supra). It is only necessary that the particular genetic engineering procedure used be capable of successfully introducing at least one gene into the host cell capable of expressing GPR 39.

After the expression cassette is introduced into the cells, the transfected cells are cultured under conditions favoring expression of GPR 39, which is recovered from the culture using standard techniques identified below.

C. GPR 39 Proteins

The isolated receptor protein can be purified from cells that naturally express it, such as from fetal brain, heart, testes, ovaries, thymus, prostate, placenta, and uterus, where expression of the receptor has been detected, especially in brain and ovaries, purified from cells that have been altered to express it (recombinant), or synthesized using known protein synthesis methods. Preferred embodiments include isolation from recombinant brain or ovarian cell lines or from diseased cells overexpressing a normal receptor gene or expressing a receptor variant. Variants that are correlated with a cancerous phenotype can be isolated from affected tissues or from at-risk individuals. Alternatively, such variants can be produced by chemical synthesis or by site-directed mutagenesis, as described above.

In one embodiment, the protein is produced by recombinant DNA techniques. For example, a nucleic acid molecule encoding the receptor polypeptide is cloned into an expression vector, the vector is introduced into a host cell and the protein is expressed in the host cell. The protein can then be isolated from the cells by an appropriate purification scheme using standard protein purification techniques.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

As is also well known, polypeptides are not always entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the amino terminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

Either naturally occurring or recombinant GPR 39 can be purified for use in functional assays. Naturally occurring GPR 39 is purified, e.g., from mammalian tissue such as brain or ovarian tissue, and any other source of a GPR 39 homolog. Recombinant GPR 39 is purified from any suitable expression system, e.g., bacterial and eukaryotic expression systems, e.g., CHO cells or insect cells.

GPR 39 may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al, supra; and Sambrook et al., supra).

A number of procedures can be employed when recombinant GPR 39 is being purified. For example, proteins having established molecular adhesion properties can be reversible fused to GPR 39. With the appropriate ligand, GPR 39 can be selectively adsorbed to a purification column and then freed from the column in a relatively pure form. The fused protein is then removed by enzymatic activity. Finally GPR 39 could be purified using immunoaffinity columns.

III. Detecting GPR 39 Modulation Using GPR-Specific Reagents

GPR 39 is an orphan G protein-coupled receptor. As the native ligand for the receptor is unknown, the present invention uses a reverse pharmacology approach to identifying GPR 39 modulators. In this approach, the orphan receptor is used as “bait” to “fish out” modulator compounds from combinatorial libraries of polypeptides, nucleic acids, immunogens and small organic molecules. The invention provides several assay approaches including direct measurement of GPR 39 protein, both total expression and compartmental distribution, and direct and indirect measurements of GPR 39 transcription.

The assays can be performed in cell-based and cell-free systems. Cell-based assays include cells naturally expressing the receptor nucleic acid or recombinant cells genetically engineered to express specific nucleic acid sequences.

The assay for GPR 39 nucleic acid expression can involve direct assay of nucleic acid levels, such as mRNA levels, or on collateral compounds involved in the signal pathway (such as cyclic AMP or phosphatidylinositol turnover). Further, the expression of genes that are up- or down-regulated in response to the receptor protein signal pathway can also be assayed. In this embodiment the regulatory regions of these genes can be operably linked to a reporter gene such as luciferase.

A. Creating Potential GPR 39-Specific Reagents

The present invention provides methods for the synthesis and preparation of compounds and compositions that modulate and/or specifically recognize the GPR 39 protein, nucleic acid, or regulatory elements controlling GPR 39 gene expression. Candidate nucleic acid-based compounds include, for example, 1) siRNAs; 2) ribozymes; 3) and antisense sequences.

Candidate protein-based compounds include, for example, 1) peptides such as soluble peptides, including fusion peptides and members of random peptide libraries (see, e.g., Lam et al., Nature 354:82-84 (1991); Houghten et al., Nature 354:84-86 (1991)) and combinatorial chemistry-derived molecular libraries made of L- and/or D-configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies, including intrabodies, as well as Fab, F(ab′).sub.2, Fab expression library fragments, and epitope-binding fragments of antibodies); and 4) small organic and inorganic molecules (e.g., molecules obtained from combinatorial and natural product libraries).

Soluble full-length receptors, or fragments of the same, that compete for ligand binding are also considered candidate reagents. Other candidate compounds include mutant receptors or appropriate fragments containing mutations that affect receptor function and thus compete for ligand. Accordingly, a fragment that competes for ligand, for example with a higher affinity, or a fragment that binds ligand but does not allow release, is encompassed by the invention. The receptor polynucleotides are also useful for constructing host cells expressing a part, or all, of the receptor polynucleotides and polypeptides.

The receptor polynucleotides are also useful for constructing transgenic animals expressing all, or a part, of the receptor polynucleotides and polypeptides. These animals are useful as model systems for GPR 39-related cancers and can be used to test compounds for their effect, through the receptor gene or gene product, on the development or progression of the disease.

Methods of preparing and employing antisense oligonucleotides, antibodies, nucleic acid probes and transgenic animals directed to the GPR 39 are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.).

1. Nucleic Acids

Nucleic acid reagents of the present invention fall into three broad categories; 1) reagents for disrupting nucleic acid processing 2) reagents for the expression of GPR 39 polynucleotides or fragments of the same, and 3) diagnostic tools for detecting GPR 39 nucleic acids.

GPR 39 nucleic acids are useful for probes, primers, and in biological assays. Where the nucleic acids are used to assess GPR 39 properties or functions, such as in the assays described herein, all or less than all of the entire cDNA can be useful. In this case, assays specifically directed to GPR functions, such as assessing agonist or antagonist activity, encompass the use of known fragments. Further, diagnostic methods for assessing receptor function can also be practiced with any fragment, including those fragments that may have been known prior to the invention.

The GPR 39-based nucleic acids discussed in this application may be obtained by methods known in the art using available materials, as discussed in detail above. For propagation and expression, GPR 39 nucleic acids are typically ligated into a suitable recombinant vector, operably linked to any necessary regulatory elements. The recombinant vector is then transfected into a suitable cell host.

Examples of suitable E. coli expression vectors that can be engineered to accept a DNA expression cassette of the present invention include pTrc (Amann et al., Gene, 69:301-315 (1988)) and pET 11d (Studier et al., Gene Expression Technology: Methods in Enzymology, 185:60-89, Academic Press, San Diego, Calif. (1990)). Examples of vectors for expression in yeast S. cerivisae include pYepSec1 (Baldari et al., EMBO J., 6:229-234 (1987)), pMFa (Kudjan and Herskowitz, Cell, 30:933-943 (1982)), pJRY88 (Schultz et al., Gene, 54:113-123 (1987)), pYES2 (Invitrogen Corporation, San Diego, Calif.), and pPicZ (Invitrogen Corp, San Diego, Calif.). Baculovirus vectors are the preferred system for expression of dsRNA's in cultured insect cells (e.g., Sf9 cells see, U.S. Pat. No. 4,745,051) and include the pAc series (Smith et al., Mol. Cell Biol., 3:2156-2165 (1983)), the pVL series (Lucklow and Summers, Virology, 170:31-39 (1989))and pBlueBac (see, e.g., U.S. Pat. Nos. 5,278,050, 5,244,805, 5,243,041, 5,242,687, 5,266,317, 4,745,051, and 5,169,784; available from Invitrogen, San Diego). For other suitable expression systems for both prokaryotic and eukaryotic cells see chapters 16 and 17 of Sambrook et al., supra. Preferred mammalian vectors are generally of viral origin and are discussed in detail below.

Infection of cells with a viral vector is a preferred method for introducing expression cassettes of the present invention into cells. The viral vector approach has the advantage that a large proportion of cells receive the expression cassette, which can obviate the need for selection of cells that have been successfully transfected. Exemplary mammalian viral vector systems include adenoviral vectors (e.g., WO 94/26914, WO 93/9191; Yei et al., Gene Therapy, 1:192-200 (1994); Kolls et al., PNAS, 91(1):215-219 (1994); Kass-Eisler et al., PNAS, 90(24):11498-502 (1993); Guzman et al., Circulation, 88(6):2838-48 (1993); Guzman et al., Cir. Res., 73(6):1202-1207 (1993); Zabner et al., Cell, 75(2):207-216 (1993); Guzman Hum Gene Ther., 4(4):403-409 (1993); Caillaud et al., Eur. J. Neurosci., 5(10):1287-1291 (1993)), adeno-associated type 1 (“AAV-1”) or adeno-associated type 2 (“AAV-2”) vectors (see WO 95/13365; Flotte et al., PNAS, 90(22):10613-10617 (1993)), hepatitis delta vectors, live, attenuated delta viruses and herpes viral vectors (e.g., U.S. Pat. No. 5,288,641), as well as vectors which are disclosed within U.S. Pat. No. 5,166,320. Other representative vectors include retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218.

Representative examples of transformation methods include using calcium phosphate precipitation (Dubensky et al., PNAS, 81:7529-7533 (1984)), direct microinjection of such nucleic acid molecules into intact target cells (Acsadi et al., Nature, 352:815-818 (1991)), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS, 89:6094 (1990)), lipofection (Felgner et al., Proc. Natl. Acad. Sci. USA, 84:7413-7417 (1989)), microprojectile bombardment (Williams et al., PNAS, 88:2726-2730 (1991)), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., Pharmac. Ther., 29:69 (1985); Curiel et al., Proc Natl Acad Sci USA, 88:8850-8854 (1991); Cotten et al., Proc Natl Acad Sci USA, 89:6094-6098 (1992); Curiel et al., Hum Gene Ther, 3:147-154 (1992); Wagner et al., Proc Natl Acad Sci USA, 89:6099-6103 (1992); Michael et al., J Biol Chem, 268:6866-6869 (1993); Curiel et al., Am J Respir Cell Mol Biol, 6:247-252 (1992); Harris et al., Am J Respir Cell Mol Biol, 9:441-447 (1993), and Friedmann et al., Science, 244:1275 (1989)), and DNA ligand (Wu et al., J. of Biol. Chem., 264:16985-16987 (1989)), as well as psoralen inactivated viruses such as AAV or Adenovirus. In one embodiment, the construct is introduced into the host cell using a liposome. Liposome based gene delivery systems are described in Debs and Zhu (1993) WO 93/24640; Mannino and Gould-Fogerite, BioTechniques, 6(7):682-691 (1988); Rose U.S. Pat. No. 5,279,833; Brigham (1991) WO 91/06309; and Felgner et al., Proc. Natl. Acad. Sci. USA, 84:7413-7414 (1987). 101061 Direct cellular uptake of oligonucleotides (whether they are composed of DNA or RNA or both) per se is presently considered a less preferred method of delivery because, in the case of siRNA and antisense molecules, direct administration of oligonucleotides carries with it the concomitant problem of attack and digestion by cellular nucleases, such as the RNAses. One preferred mode for administration of the expression cassettes of the present invention takes advantage of known vectors to facilitate the delivery of the expression cassette such that it will be expressed by the desired target cells. Such vectors include plasmids and viruses (such as adenoviruses, retroviruses, and adeno-associated viruses) [and liposomes] and modifications therein (e.g., polylysine-modified adenoviruses [Gao et al., Human Gene Therapy, 4:17-24 (1993)], cationic liposomes [Zhu et al., Science, 261:209-211 (1993)] and modified adeno-associated virus plasmids encased in liposomes [Phillip et al., Mol. Cell. Biol., 14:2411-2418 (1994)], as described supra.

a) siRNA

siRNA molecules are small (typically 16-25 bp) double-stranded RNAs that elicit a process known as RNA interference (RNAi), a form of sequence-specific gene inactivation. A proposed mechanism for RNAi action proposes an ATP-dependent cleavage of mRNA molecules activated by a short double-stranded RNA. The nucleotide sequence of the cleaved mRNA molecules are reported to contain a sequence fragment homologous to that of the double-stranded RNA. Zamore, Phillip et al., (2000) Cell, 101:25-33. RNA interference has been shown to exist in mammalian cell lines, oocytes, early embryos and some cell types (see e.g., Elbashir, Sayda M., et al. (2001) Nature 411:494-497). The development of efficient methods for screening effective siRNAs offers a means for identifying the functional characteristics of genes targeted by such siRNAs, through a process of subtractive phenotypic analysis, a technology developed by the Assignee hereof known as Inverse Genomics™. Thus by creating and expressing in a cancer cell siRNAs specific for sequence(s) found in GPR 39 mRNA, GPR 39 expression can be down regulated.

siRNAs for use in the present invention can be produced from a GPR-39-encoding nucleic acid sequence. For example, short complementary DNA strands are first prepared that represent portions of both the “sense” and “antisense” strands of the GPR 39 coding region. This is typically accomplished using solid phase nucleic acid synthesis techniques, as detailed above. The short duplex DNA thus formed is ligated into a suitable vector that is then used to transfect a suitable cell line. Other methods for producing siRNA molecules targeted to GPR 39 are known in the art. (See, e.g., Elbashir, S. M., Harborth, J., Lendeckel, W., Yalcin, A., Weber, K., and Tuschl, T. (2001) Duplexes of 21-nucleotide RNAs mediate RNA interference in cultured mammalian cells. Nature 411:494-498). Libraries of siRNAs specific for GPR 39 can be constructed by, for example, mechanically shearing GPR 39 cDNA, ligating the resulting fragments into suitable vector constructs and transfecting a suitable host cell with the vectors.

For a review of RNAi and siRNA expression, see Hammond, Scott M. et al., Nature Genetics Reviews, 2:110-119; Fire, Andrew (1999) TIG, 15(9):358-363; Bass, Brenda L. (2000) Cell, 101:235-238.

b) Antisense Sequences

The targeting of antisense oligonucleotides to mRNA is another mechanism to shut down protein synthesis, and, consequently, represents a powerful and targeted approach to knocking out GPR 39 expression. For example, the synthesis of polygalactauronase and the muscarine type 2 acetylcholine receptor are inhibited by antisense oligonucleotides directed to their respective mRNA sequences (U.S. Pat. Nos. 5,739,119 and 5,759,829, each specifically incorporated herein by reference in its entirety). Further, examples of antisense inhibition have been demonstrated with the nuclear protein cyclin, the multiple drug resistance gene (MDG1), ICAM-1, E-selectin, STK-1, striatal GABA.sub.A receptor and human EGF (Jaskulski et al., 1988; Vasanthakumar and Ahnmed, 1989; Peris et al., 1998; U.S. Pat. Nos. 5,801,154; 5,789,573; 5,718,709 and 5,610,288, each specifically incorporated herein by reference in its entirety). Antisense constructs have also been described that inhibit and can be used to treat a variety of abnormal cellular proliferations, e.g. cancer (U.S. Pat. Nos. 5,747,470; 5,591,317 and 5,783,683, each specifically incorporated herein by reference in its entirety).

The invention provides therefore oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of a GPR 39 mRNA.

Selection of antisense compositions specific for a given gene sequence is based upon analysis of the chosen target sequence (i.e. in these illustrative examples the rat and human sequences) and determination of secondary structure, T.sub.m, binding energy, relative stability, and antisense compositions were selected based upon their relative inability to form dimers, hairpins, or other secondary structures that would reduce or prohibit specific binding to the target mRNA in a host cell.

Highly preferred target regions of the mRNA, are those which are at or near the AUG translation initiation codon, and those sequences which were substantially complementary to 5′ regions of the mRNA. These secondary structure analyses and target site selection considerations were performed using v.4 of the OLIGO primer analysis software (Rychlik, 1997) and the BLASTN 2.0.5 algorithm software (Altschul et al., 1997).

The invention also encompasses vectors in which aGPR 39 nucleic acid is cloned into a vector in reverse orientation, but operably linked to a regulatory sequence that permits transcription of antisense RNA. Thus, an antisense transcript can be produced to all, or to a portion, of the nucleic acid sequences described herein, including both coding and non-coding regions. Expression of this antisense RNA is subject to each of the parameters described above in relation to expression of the sense RNA (regulatory sequences, constitutive or inducible expression, tissue-specific expression).

Antisense nucleic acids maybe obtained from libraries encoding GCP 39 or synthesized synthetically. Transfection of suitable host cells with GPR 39 is performed in a manner analogous to that described for siRNAs above.

c) Ribozymes

The GPR 39 coding sequence is also useful for designing ribozymes corresponding to all, or a part, of the mRNA produced from genes encoding the polynucleotides described herein. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., 1992). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. (1992). Examples of hairpin motifs are described by Hampel et al. (Eur. Pat. Appl. Publ. No. EP 0360257), Hampel and Tritz (1989), Hampel et al. (1990) and U.S. Pat. No. 5,631,359 (specifically incorporated herein by reference). An example of the hepatitis 6 virus motif is described by Perrotta and Been (1992); an example of the RNaseP motif is described by Guerrier-Takada et al. (1983); Neurospora VS RNA ribozyme motif is described by Collins (Saville and Collins, 1990; Saville and Collins, 1991; Collins and Olive, 1993); and an example of the Group I intron is described in (U.S. Pat. No. 4,987,071, specifically incorporated herein by reference). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Small enzymatic nucleic acid motifs (e.g., of the hammerhead or the hairpin structure) may also be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g., Qi-Xiang et al., Nucl. Acid Res., 28:13, p. 2605-2612 (2000)). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference).

Ribozymes may be added directly, or can be complexed with cationic lipids, lipid complexes, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Hammerhead or hairpin ribozymes may be individually analyzed by computer folding (Jaeger et al., 1989) to assess whether the ribozyme sequences fold into the appropriate secondary structure. Those ribozymes with unfavorable intramolecular interactions between the binding arms and the catalytic core are eliminated from consideration. Varying binding arm lengths can be chosen to optimize activity. Generally, at least 5 or so bases on each arm are able to bind to, or otherwise interact with, the target RNA.

Ribozymes of the hammerhead or hairpin motif may be designed to anneal to various sites in the mRNA message, and can be chemically synthesized. The method of synthesis used follows the procedure for normal RNA synthesis as described in Usman et al. (1987) and in Scaringe et al. (1990) and makes use of common nucleic acid protecting and coupling groups, such as dimethoxytrityl at the 5′-end, and phosphoramidites at the 3′-end. Average stepwise coupling yields are typically >98%. Hairpin ribozymes may be synthesized in two parts and annealed to reconstruct an active ribozyme (Chowrira and Burke, 1992). Ribozymes may be modified extensively to enhance stability by modification with nuclease resistant groups, for example, 2′-amino, 2′-C-ally, 2′-flouro, 2′-o-methyl, 2′-H (for a review see e.g., Usman and Cedergren, 1992). Ribozymes may be purified by gel electrophoresis using general methods or by high pressure liquid chromatography and resuspended in water.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perrault et al, 1990; Pieken et al., 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

2. Proteins

The invention also includes protein and peptide reagents capable of modulating GPR 39 expression, or detection of the GPR 39 protein in subcellular fractions.

Proteins and polypeptides of the present invention may be isolated from native sources or, where the nucleic acid encoding the polypeptide is available, produced using recombinant methods well known in the art and discussed in the references cited above. When polypeptide reagents are produced recombinantly and not secreted into the medium, the protein can be isolated from the host cell by standard disruption procedures, including freeze thaw, sonication, mechanical disruption, use of lysing agents and the like. Regardless of source, the polypeptide can be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides, the polypeptides can have various glycosylation patterns, depending upon the cell, or maybe non-glycosylated as when produced in bacteria. In addition, the polypeptides may include an initial modified methionine in some cases as a result of a host-mediated process.

a) Antibodies

Methods of producing polyclonal and monoclonal antibodies that react specifically with GPR 39 are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of GPR 39 comprising immunogens may be used to produce antibodies specifically reactive with GPR 39. For example, recombinant GPR 39 or an antigenic fragment thereof, is isolated as described herein. Recombinant protein can be expressed in eukaryotic or prokaryotic cells as described above, and purified as generally described above. Recombinant protein is one embodiment of an immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to GPR 39. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-GPR 39 proteins or even other related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better. 101331 Once GPR 39 specific antibodies are available, GPR 39 can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ³⁵S or ³H. Additional labels are discussed at the end of this section, below.

The antibodies are also useful for inhibiting receptor function, for example, blocking ligand binding. These uses can also be applied in a therapeutic context in which treatment involves inhibiting receptor function. An antibody can be used, for example, to block ligand binding. Antibodies can be prepared against specific fragments containing sites required for function or against intact receptor associated with a cell.

b) Intrabodies

Intrabodies are engineered antibodies that can be expressed within a cell and target an intracellular molecule of molecular domain. Using this technique, intracellular signals and enzyme activities can be inhibited, or their transport to cellular compartments prevented [Marasco, W. A., et al., Proc. Natl. Acad. Sci. USA 90:7889-7893 (1993)]. In particular, intrabodies directed against GPR 39 will bind to the nascent GPR 39 protein and direct it to the ubiquitin pathway for catalytic degradation, rather than to the cellular membrane. Thus intrabodies provide yet another approach to down regulating GPR 39 expression and activity.

The intrabody method is analogous to the inactivation of proteins by deletion or mutation, but is directed at the level of gene product rather than at the gene itself. Using the intrabody strategy even molecules involved in essential cellular pathways can be targeted, modified or blocked. Antibody genes for intracellular expression can be derived either from murine or human monoclonal antibodies or from phage display libraries. For intracellular expression small recombinant antibody fragments, containing the antigen recognizing and binding regions, can be used. Intrabodies can be directed to different intracellular compartments by targeting sequences attached to the antibody fragments.

The construction and use of intrabodies is discussed in U.S. Pat. No. 6,004,940, which is incorporated herein by reference.

c) Peptides

Combinatorial peptide libraries can be screened to identify antagonists of GPR 39. Combinatorial peptide libraries can be constructed from genomic or cDNA libraries, or by using non-cellular synthetic methods. Techniques for solid phase synthesis are described by Barany and Merrifield, Solid-Phase Peptide Synthesis, pp. 3-284 in The Peptides: Analysis, Synthesis, Biology. Vol. 2: Special Methods in Peptide Synthesis, Part A.; Merrifield, et al., J. Am. Chem. Soc. 85: 2149-2156 (1963), and Stewart et al., Solid Phase Peptide Synthesis, 2nd ed., Pierce Chem. Co., Rockford, III. (1984). Proteins may be synthesized by condensation of the amino and carboxy termini of shorter fragments. Methods of forming peptide bonds by activation of a carboxy terminal end (e.g., by the use of the coupling reagent N,N′-dicycylohexylcarbodiimide) are known to those of skill.

The proteins useful in this invention may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503.

Peptide and protein reagents can optionally labeled, as described below, or may be used in the screening assays of the present invention to ascertain their ability to modulate GPR 39 expression or activity.

d) GPR 39 Receptors and Receptor Fragments

GPR 39 polypeptides are useful in competition binding assays in methods designed to discover compounds that interact with the receptor. Thus, a compound is exposed to a receptor polypeptide under conditions that allow the compound to bind or to otherwise interact with the polypeptide. Soluble receptor polypeptide is also added to the mixture. If the test compound interacts with the soluble receptor polypeptide, it decreases the amount of complex formed or activity from the receptor target. This type of assay is particularly useful in cases in which compounds are sought that interact with specific regions of the receptor. Thus, the soluble polypeptide that competes with the target receptor region is designed to contain peptide sequences corresponding to the region of interest.

Particularly preferred peptides include homologous sequences and/or fragments of a transmembrane domain of one or more GPCRs or homologs thereof. For purposes of creating combinatorial libraries, any and all domains of any or all know GPCRs may serve as model primary sequences for construction of the library. Preferably members of the library will comprise at least 15, preferably 20, more preferably 23 amino acids, although longer peptides are also contemplated where particular intra or extracellularly located sequences are desired. For information regarding the effects on cell signally caused by alterations of the transmembrane domain motif og GPCRs, see Schoneberg et al., EMBO J. 15:1283(1996); Wong et al., J. Biol. Chem., 265:6219 (1990); Monnot et al., J. Biol. Chem., 271:1507 (1996); Gudermann et al., Annu. Rev. Neurosci., 20:399 (1997); Osuga et al., J. Biol. Chem., 272:25006(1997); and Hebert et al., J. Biol. Chem., 271(27):16384-92 (1996).

e) Small Organic Molecules

The compounds tested as modulators of GPR 39 can be any small chemical compound, or a biological entity, such as a protein, sugar, nucleic acid or lipid. Screening combinatorial libraries of small organic molecules offers an approach to identifying useful therapeutic compounds or precursors targeted to GP 39. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual, therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

3. Labels for Proteins and Nucleic Acids

The particular label or detectable group used in the assay is not a critical aspect of the invention, as long as it does not significantly interfere with the specific binding of the antibody or protein used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™); fluorescent dyes and techniques capable of monitoring the change in fluorescent intensity, wavelength shift, or fluorescent polarization (e.g., fluorescein isothiocyanate, Texas red, rhodamine, and the like); radiolabels (e.g., ³H, ¹²⁵I, ³⁵S, ¹⁴C, or ³²P); enzymes (e.g., horse radish peroxidase, alkaline phosphatase and others commonly used in an ELISA); and colorimetric labels such as colloidal gold or colored glass or plastic beads (e.g., polystyrene, polypropylene, latex, etc.). For exemplary methods for incorporating such labels, see U.S. Pat. Nos. 3,940,475; 3,817,837; 3,850,752; 3,939,350; 3,996,345; 4,277,437; 4,275,149; and 4,366,241.

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), that is either inherently detectable or covalently bound to a signal system, such as a detectable enzyme, a fluorescent compound, or a chemiluminescent compound. The ligands and their targets can be used in any suitable combination with antibodies that recognize GRP 39, or secondary antibodies that recognize anti-GRP 39 antibodies. Other possibilities for indirect labeling include biotinylation of one constituent followed by binding to avidin coupled to one of the above label groups.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge-coupled devices (CCDs) or photomultipliers and the like. Similarly, enzymatic labels may be detected by providing the appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

B. High Throughput Pre-Screening

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

Antibodies (excluding intrabodies), peptides and small organic molecules all lend themselves to high throughput pre-screening as described herein; ribozymes, antisense sequences, intrabodies and siRNAs however do not. The difference between the two groups is the result of the former group acting as ligands capable of interacting with the GPR 39 protein directly, whereas the latter group does not. (intrabodies may recognize the cytoplasmic domain(s) of the GPR 39 protein, but this portion of the molecule is frequently inaccessable in the pre-screening assays). As the pre-screening assays use the GPR 39 protein as the “bait” to “fish out” prospective ligands, the assays simply are not designed to detect expected mechanisms of GPR 39 modulation by antisense sequences, ribozymes, siRNAs and most intrabodies. Activity and expression assays amenable to this latter group of potential GPR 39 modulators are discussed below.

1. High Throughput Assays of Chemical Libraries

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput assays. Thus, for example, Zymark Corp. provides technical bulletins describing screening systems for detecting the modulation of gene transcription, ligand binding, and the like.

2. C. Solid State and Soluble High Throughput Assays

To perform cell-free drug screening assays, it is desirable to immobilize either the receptor protein, or fragment, or its target molecule to facilitate separation of complexes from uncomplexed forms of one or both of the proteins, as well as to accommodate automation of the assay.

In one embodiment the invention provides soluble assays using molecules such as a ligand binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; GRP 39; or a cell or tissue expressing GRP 39, either naturally occurring or recombinant. In another embodiment, the invention provides solid phase based in vitro assays in a high throughput format, where the domain, chimeric molecule, GRP 39, or cell or tissue expressing GRP 39 is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 different compounds is possible using the integrated systems of the invention. More recently, microfluidic approaches to reagent manipulation have been developed, e.g., by Caliper Technologies (Palo Alto, Calif.).

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the taste transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Another approach uses recombinant bacteriophage to produce large libraries. Using the “phage method” (Scott and Smith, Science 249:386-390, 1990; Cwirla, et al, Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In another aspect, synthetic libraries (Needels et al, Proc. Natl. Acad. Sci. USA 90:10700-4, 1993; Ohlmeyer et al., Proc. Natl. Acad. Sci. USA 90:10922-10926, 1993; Lam et al., International Patent Publication No. WO 92/00252; Kocis et al., International Patent Publication No. WO 9428028) and the like can be used to screen for GPR 39 ligands according to the present invention.

The screening can be performed with recombinant cells that express the GPR 39, or alternatively, using purified protein, e.g., produced recombinantly, as described above. For example, the ability of labeled, soluble or solubilized GPR 39 that includes the ligand-binding portion of the molecule, to bind ligand can be used to screen libraries, as described in the foregoing references. In a specific embodiment, infra, cell membranes containing recombinantly produced GPR 39 (both long and short forms) were used in binding assays with various ligands.

Radioligand binding assays allow further characterization of hits from high throughput screens as well as analogs of neurotensin agonists and antagonists. Using membranes from cells stably expressing each neurotensin receptor subtype, one point binding assays are first performed to determine how well a particular concentration, such as 25 .mu.M, of each hit or analog displaces specific [³H] NT binding from the receptor. If the hit or analog displaces ≧50% of the [³H] NT bound, a competition binding assay is performed. Competition binding assays, as shown in the Examples, infra, evaluate the ability of increasing concentrations of competitor (the hit or any test compound analog) to displace [³H] NT binding at each neurotensin receptor subtype. The resulting K₁ value indicates the relative potency of each hit or test compound for a particular receptor subtype. These competition binding assays allow the determination of the relative potencies of each hit or test compound at a particular receptor subtype, as well as to determine the receptor subtype selectivity of each hit or test compound.

3. Computer-Based Assays

Yet another assay for compounds that modulate GRP 39 activity involves computer assisted drug design, in which a computer system is used to generate a three-dimensional structure of GRP 39 based on the structural information encoded by the amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering protein amino acid sequences of at least 10 amino acid residues or corresponding nucleic acid sequences encoding a GRP 39 polypeptide into the computer system. The amino acid sequence of the polypeptide listed as SEQ ID: 2, and conservatively modified versions thereof can be used for this purpose. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the GRP 39 protein to identify ligands that bind to GRP 39. Binding affinity between the protein and ligands is determined using energy terms to determine which ligands have an enhanced probability of binding to the protein.

C. Activity and Expression Assays

GPR 39 and its alleles and polymorphic variants are G-protein coupled receptors that have been observed to be elevated in certain cancers. The activity of GPR 39 polypeptides can be assessed using a variety of in vitro and in vivo assays that determine functional, physical and chemical effects, e.g., measuring ligand binding (e.g., by radioactive ligand binding), second messengers (e.g., cAMP, cGMP, IP₃, DAG, or Ca²⁺), ion flux, phosphorylation levels, transcription levels, neurotransmitter levels, and the like. Furthermore, such assays can be used to test for inhibitors and activators of GPR 39. Modulators can also be genetically altered versions of GPR 39. Such modulators are useful in the treatment and diagnosis of cancer.

The GPR 39 of the assay will be selected from a polypeptide having a sequence of SEQ ID NO: 2 or conservatively modified variant thereof. Alternatively, the GPR 39 of the assay will be derived from a eukaryote and include an amino acid subsequence were the homology will be at least 60%, preferably at least 75%, more preferably at least 90% and most preferably between 95% and 100% that of SEQ ID NO: 2. Optionally, the polypeptide of the assays will comprise a domain of GPR 39, such as an extracellular domain, transmembrane domain, cytoplasmic domain, ligand binding domain, subunit association domain, active site, and the like. Either GPR 39 or a domain thereof can be covalently linked to a heterologous protein to create a chimeric protein used in the assays described herein.

Modulators of GPR 39 activity are tested using GPR 39 polypeptides as described above, either recombinant or naturally occurring. The protein can be isolated, expressed in a cell, expressed in a membrane derived from a cell, expressed in tissue or in an animal, either recombinant or naturally occurring.

Receptor-G-protein interactions can also be examined. For example, binding of the G-protein to the receptor or its release from the receptor can be examined. For example, in the absence of GTP, an activator will lead to the formation of a tight complex of a G protein (all three subunits) with the receptor. This complex can be detected in a variety of ways, as noted above. Such an assay can be modified to search for inhibitors. Add an activator to the receptor and G protein in the absence of GTP, form a tight complex, and then screen for inhibitors by looking at dissociation of the receptor-G protein complex. In the presence of GTP, release of the alpha subunit of the G protein from the other two G protein subunits serves as a criterion of activation.

Activated GPCR receptors become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G-proteins. The kinase/arrestin pathway plays a key role in the desensitization of many GPCR receptors. For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu. Rev. Biochem. 67:653-92 (1998).

Samples or assays that are treated with a potential GPR 39 inhibitor or activator are compared to control samples without the test compound, to examine the extent of modulation. Control samples (untreated with activators or inhibitors) are assigned a relative GPR 39 activity value of 100. Inhibition of GPR 39 is achieved when the GPR 39 activity value relative to the control is about 90%, preferably 50%, more preferably 25-0%. Activation of GPR 39 is achieved when the GPR 39 activity value relative to the control is 110%, preferably 150%, 200-500%, or 1000-2000%.

Changes in ion flux may be assessed by determining changes in polarization (i.e., electrical potential) of the cell or membrane expressing GPR 39. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., i J. Pharmacol. Meth. 25:185-193 (1991); Holevinsky et al., J. Membrane Biology 137:59-70 (1994)). Generally, the compounds to be tested are present in the range from 1 pM to 100 mM.

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP3 or cAMP.

Assays for G-protein coupled receptors include cells that are loaded with ion or voltage sensitive dyes to report receptor activity. Assays for determining activity of such receptors can also use known agonists and antagonists for other G-protein coupled receptors as negative or positive controls to assess activity of tested compounds. In assays for identifying modulatory compounds (e.g., agonists, antagonists), changes in the level of ions in the cytoplasm or membrane voltage will be monitored using an ion-sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα15 and Gα16 can be used in the assay of choice (Wilkie et al., Proc. Nat'l Acad. Sci. USA 88:10049-10053 (1991)). Such promiscuous G-proteins allow coupling of a wide range of receptors.

In one embodiment, GPR 39 activity is measured by expressing GPR 39 in a heterologous cell with a promiscuous G-protein that links the receptor to a phospholipase C signal transduction pathway (see Offermanns & Simon, J. Biol. Chem. 270:15175-15180 (1995)). Optionally the cell line is HEK-293 (which does not naturally express GPR 39) and the promiscuous G-protein is Ga1 5 (Offermanns & Simon, supra). Modulation of GPR 39 activation is assayed by measuring changes in intracellular Ca²⁺ levels, which change in response to modulation of the GPR 39 signal transduction pathway via administration of a molecule that associates with GPR 39. Changes in Ca²⁺ levels are optionally measured using fluorescent Ca²⁺ indicator dyes and fluorometric imaging.

In one embodiment, the changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of epm in the presence of antagonist to cpm in the presence of buffer control (which may or may not contain an agonist).

In another embodiment, transcription levels can be measured to assess the effects of a test compound on signal transduction. A host cell containing the protein of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)). [01921 The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

Particularly preferred assays of the present invention are discussed individually, below.

1. Suitable Cell Systems

The choice of cell system is critical to the success of the assay performed, as cell lines with a good history of GPR expression containing a wide repertoire of G-proteins allow functional coupling to downstream effectors. For expression of GPR 39cell lines of choice include, P8R3 and its parent cell line PA1, SKBr3, HT 1080, MCF-7, HeLa, A549 and CHO cells. pA1 and SKBr3 are particularly preferred cell lines for GPR39 expression systems.

Methods of transfecting cells e.g. mammalian cells, with such nucleic acid to obtain cells in which the receptor is expressed on the surface of the cell are well known in the art. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

The receptor polynucleotides can also be expressed by expression vectors that are operative in yeast. Examples of vectors for expression in yeast e.g., S. cerevisiae include pYepSec1 (Baldari, et al., EMBO J. 6:229-234 (1987)), pMFa (Kudjan et al., Cell 30:933-943(1982)), pJRY88 (Schultz et al., Gene 54:113-123 (1987)), and pYES2 (Invitrogen Corporation, San Diego, Calif.). Insect cells are another potential expression system, for example, baculovirus expression vectors. Baculovirus vectors available for expression of proteins in cultured insect cells (e.g., Sf9 cells) include the pAc series (Smith et al., Mol. Cell Biol. 3:2156-2165 (1983)) and the pVL series (Lucklow et al., Virology 170:31-39 (1989)).

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2.sup.nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the receptor polynucleotides can be introduced either alone or with other polynucleotides that are not related to the receptor polynucleotides such as those providing trans-acting factors for expression vectors. When more than one vector is introduced into a cell, the vectors can be introduced independently, co-introduced or joined to the receptor polynucleotide vector.

It may be desirable to express the polypeptide as a fusion protein. Accordingly, the invention provides fusion vectors that allow for the production of the receptor polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression can be maximized in a host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990) 119-128). Alternatively, the sequence of the polynucleotide of interest can be altered to provide preferential codon usage for a specific host cell, for example E. coli. (Wada et al., Nucleic Acids Res. 20:2111-2118 (1992)).

Transfected cells may also be used to test compounds and screen compound libraries, such as those described above, to obtain compounds which bind to the GPR 39 receptor, as well as compounds which activate or inhibit activation of functional responses in such cells, and therefore are likely to do so in vivo. (See, for example, U.S. Pat. Nos. 5,053,337; 5,155,218; 5,360,735; 5,472,866; 5,476,782; 5,516,653; 5,545,549; 5,556,753; 5,595,880; 5,602,024; 5,639,652; 5,652,113; 5,661,024; 5,766,879; 5,786,155; and 5,786,157, the disclosures of which are hereby incorporated by reference in their entireties into this application.)

Host cells are also useful for conducting cell-based assays involving the receptor or receptor fragments. Thus, a recombinant host cell expressing a native receptor is useful to assay for compounds that stimulate or inhibit receptor function. This includes ligand binding, gene expression at the level of transcription or translation, G-protein interaction, and components of the signal transduction pathway.

Recombinant host cells are also useful for expressing the chimeric polypeptides described herein to assess compounds that activate or suppress activation by means of a heterologous amino terminal extracellular domain (or other binding region). Alternatively, a heterologous region spanning the entire transmembrane domain (or parts thereof) can be used to assess the effect of a desired amino terminal extracellular domain (or other binding region) on any given host cell. In this embodiment, a region spanning the entire transmembrane domain (or parts thereof) compatible with the specific host cell is used to make the chimeric vector. Alternatively, a heterologous carboxy terminal intracellular, e.g., signal transduction, domain can be introduced into the host cell.

Binding and/or activating compounds can also be screened by using recombinant cells having chimeric receptor proteins in which the amino terminal extracellular domain, or parts thereof, the entire transmembrane domain or subregions, such as any of the seven transmembrane segments or any of the intracellular or extracellular loops and the carboxy terminal intracellular domain, or parts thereof, can be replaced by heterologous domains or subregions. For example, a G-protein-binding region can be used that interacts with a different G-protein then that which is recognized by the native receptor. Accordingly, a different set of signal transduction components is available as an end-point assay for activation. Alternatively, the entire transmembrane portion or subregions (such as transmembrane segments or intracellular or extracellular loops) can be replaced with the entire transmembrane portion or subregions specific to a host cell that is different from the host cell from which the amino terminal extracellular domain and/or the G-protein-binding region are derived. This allows for assays to be performed in other than the specific host cell from which the receptor is derived. Alternatively, the amino terminal extracellular domain (and/or other ligand-binding regions) could be replaced by a domain (and/or other binding region) binding a different ligand, thus, providing an assay for test compounds that interact with the heterologous amino terminal extracellular domain (or region) but still cause signal transduction. Finally, activation can be detected by a reporter gene containing an easily detectable coding region operably linked to a transcriptional regulatory sequence that is part of the native signal transduction pathway.

2. Transgenic Animals

Drug screening assays can also be performed in transgenic animal models such as those described herein. Thus, naturally-occurring mutants or mutants made in a laboratory can be used to create transgenic animals that serve as a basis for drug screening. This model is particularly useful in assessing the total effect of an in vivo environment on the effect of a given drug. These animals can serve as an animal model for disease, such as various forms of cancer and cardiovascular diseases, so that in addition to ascertaining an effect on the specific mutant, an effect can be ascertained on the total system. Transgenic animals can thus be created that overexpress the receptor protein or express a variant, leading to, for example, loss of contact inhibition. Alternatively, such animals can be created that underexpress the receptor or, in the case of “knockout” mice, lack one or more copies of the gene. Variant genes include modifications such as insertion, deletion, and nucleotide substitutions. In one embodiment, gene expression is under the control of an inducible promoter. Therefore, modulation of the gene and, thus, modulation of the disease state is provided. The invention thus also encompasses cardiomyocytes derived from transgenic animals in which the cardiovascular disease has been produced by means of expression of the receptor in recombinant host cells, is naturally occurring, or occurs as the result of other protocols and/or agents.

This invention further provides a transgenic, nonhuman mammal expressing DNA encoding a mammalian GPR 39 receptor in accordance with this invention. This invention provides a transgenic, nonhuman mammal comprising a homologous recombination knockout of a native mammalian GPR 39 receptor. This invention further provides a transgenic, nonhuman mammal whose genome comprises antisense DNA complementary to DNA encoding a mammalian GPR 39 receptor in accordance with this invention so placed within such genome as to be transcribed into antisense mRNA which is complementary and hybridizes with mRNA encoding the mammalian GPR 39 receptor so as to thereby reduce translation or such mRNA and expression of such receptor. In one embodiment, the DNA encoding the mammalian GPR 39 receptor additionally comprises an inducible promoter. In another embodiment, the DNA encoding the mammalian GPR 39 receptor additionally comprises tissue specific regulatory elements. In another embodiment, the transgenic, nonhuman mammal is a mouse.

Other examples of transgenic animals include non-human primates, sheep, dogs, cows, goats, chickens, and amphibians.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No.4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

3. Assay Methodologies

a) Reporter Genes

The practice of using a reporter gene to analyze nucleotide sequences that regulate transcription of a gene-of-interest is well documented. The demonstrated utility of a reporter gene is in its ability to define domains of transcriptional regulatory elements of a gene-of-interest. Reporter genes express proteins that serve as detectable labels indicating when the control elements regulating reporter gene expression are up or down-regulated in response to outside stimuli.

By way of example, two types of reporter gene assay are discussed below. The first is a scorable reporter gene, whose expression can be quantified, giving a proportional indication of the level of expression supported by the genetic construct comprising the reporter gene. The second example is a selectable reporter gene. When expressed, the selectable reporter gene allows the host cell harboring the reporter gene to survive under restrictive conditions that would otherwise kill (or retard the growth of) the host cell.

Scorable reporter genes are typically used when the relative activity of a genetic construct is sought, whereas selectable reporters are used when confirmation of the presence of the reporter expression construct within the cell is desired.

Scorable Markers—The Luciferase Assay

Firefly luciferase expression systems have become widely used for quantitative analysis of transcriptional modulation in living cells (see, e.g., Wood, K. V. (1998) Promega Notes 65:14). In particular, recombinant cells comprising this reporter construct enable libraries of small molecules to be rapidly screened for those affecting specific aspects of cellular physiology, such as receptor function or intracellular signal transduction. deWet et al. (1987) Mol. Cell. Biol. 7:725; Wood, K. V. (1991) In: Bioluminescence and Chemiluminescence: Current Status, eds. P. Stanley and L. Kricka, John Wiley and Sons, Chichester, 11.

The luciferase assay could be used to screen any of the potential GPR-specific reagents listed above. For example, by placing the luciferase gene under the control of the GPR 39 promoter, reagents that bind to the GPR 39 protein can trigger a feedback loop modulating expression of the luciferase gene. Similarly, by creating a fusion protein comprising the luciferase and GPR 39 coding sequences, siRNAs, Antisense sequences and ribozymes targeted against the GPR 39 gene can be screened, as any reagent acting on the GPR 39 transcript will necessarily disrupt expression of the luciferase enzyme encoded in the same transcript.

Modulators will manifest themselves by altering the amount of light emitted by the luciferase-catalyzed hydrolysis of ATP, with up-modulators increasing the amount of light emitted (they induce increased luciferase production) and down-modulators decreasing the amount of light emitted (by inhibiting luciferase production) in proportion to the degree of expressional modulation (at least within the linear range limits of the assay). Luciferase assay kits and other reporter gene constructs suitable for use in the present invention are well known in the art and commercially available, e.g., Invitrogen and Promega. See, e.g., Steady-GloTM Luciferase Assay Reagent Technical Manual Luciferase Assay Reagent Technical Manual #TM05 1, Promega Corporation.

Selectable Marker Assay

A number of selectable marker systems can be used in the present invention, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

Typically, selectable markers are included in expression cassettes comprising the target gene to or construct to be incorporated into the host cell. The selectable marker may be under the control of the same promoter as the target construct, e.g., as part of a fusion protein or polycistronic transcript; or may be under the control of an independent promoter.

As suggested above, the purpose of the selectable marker is to confer selectable growth characteristics on cells that are able to express it. By including the selectable marker in the same nucleic acid comprising the target gene or construct, the selectable marker will be included in any cell transformed with the target. Therefore, by selecting for the growth characteristics conferred by the selectable marker, cells transfected with the target can be selected.

In some instances genes are only expressed under particular conditions in particular cell types. E.g., certain gene products are only associated with tumorigenic forms of the cell. These gene products are termed neoplastic markers. When neoplastic marker genes have been identified and isolated, the ability to regulate them can be studied by placing a selectable marker under the control of the neoplastic gene promoter. As an example, BRCA-1 is a tumor suppressor that is normally constitutively expressed, but known to be expressed at very low or undetectable levels in certain forms of breast cancer. (Miki et al., Science 266: 66-71, 1994). Often, and perhaps always, breast cancers expressing low levels of BRCA-1 also display elevated levels of GPR 39. To study the linkage between elevated GPR 39 expression and depressed BRCA-1 expression, a hygro gene is operably linked to a BRCA-1 promoter and transfected into breast cancer cells displaying the appropriate phenotype. The transfected cells are then treated with a compound known to specifically bind and down regulate GPR 39. Subsequently the treated, transfected cells are plated into media containing hygromycin in parallel with control cells that have been transfected with the hygro selectable marker but have not been treated with the compound that specifically binds GPR 39. Only treated cells will grow in the presence of hygromycin. Subsequent analysis of GPR 39 and BRCA-1 expression will reveal that the hygromycin resistant cells treated with the compound known to specifically bind and down regulate GPR 39 have down-regulated GPR 39 expression, and up-regulated BRCA-1, and display a normal cell phenotype.

b) PCR-Based Assays

(1) Quantitative PCR

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988); and Nakazawa et al., PNAS 91:360-364 (1994)), the latter of which can be particularly useful for detecting point mutations in the gene (see Abravaya et al., Nucleic Acids Res. 23:675-682 (1995)). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

(2) Real Time PCR

Real-time PCR assays take advantage of those cycles of a normal PCR reaction where the DNA being amplified is increasing at a logrythmic rate and hence proportional to the amount of DNA present. Several kits are commercially available for performing real-time PCR. One such kit is the TaqMan assay.

The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time. In an alternative homogeneous hybridization based procedure, molecular beacons are used for allele discriminations. Molecular beacons are hairpin-shaped oligonucleotide probes that report the presence of specific nucleic acid molecules in homogeneous solutions. When they bind to their targets they undergo a conformational reorganization that restores the fluorescence of an internally quenched fluorophore. (See, e.g., Heid, C. A., Stevens, J., Livak, K. J. and Williams, P. M. Real time quantitative PCR. Genome Res. 6:986-994 (1996); Gibson, U. E. M., Heid, C. A. and Williams, P. M. A novel method for real time quantitative RT-PCR. Genome Res. 6:995-1001 (1996)).

c) Measures of Expressed Nucleic Acids and Proteins

(1) Northern Blotting

Northern blot methods allow RNA isolated from cells of interest to be separated using gel electrophoresis techniques. After separation, nucleic acids are transferred to membranes and hybridized with radio-labeled nucleotide probes. For analysis of expression maps, poly A (adenylyl) probed are used, which hybridize to mRNA species present on the blot.

The present invention uses both traditional and expression map Northern blotting. Expression of GPR 39 and other genes of interest can be tracked using probes specific for these genes. Expression mapping can be used to monitor alterations in gene expression in response to GPR 39-specific binding agents.

Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996.

(2) Global Expression Profiling

Through the use of high density oligonucleotide arrays, expression profiles for individual cells can be rapidly obtained and compared. High density arrays are particularly useful for monitoring expression control at the transcriptional, RNA processing and degradation level. The fabrication and application of high density arrays in gene expression monitoring have been disclosed previously in, for example, WO 97/10365, WO 92/10588, U.S. Pat. No. 6,040,138 incorporated herein for all purposes by reference. In some embodiments using high density arrays, high density oligonucleotide arrays are synthesized using methods such as the Very Large Scale Immobilized Polymer Synthesis (VLSIPS) disclosed in U.S. Pat. No. 5,445,934. Each oligonucleotide occupies a known location on a substrate. A nucleic acid target sample is hybridized with a high density array of oligonucleotides and then the amount of target nucleic acids hybridized to each probe in the array is quantified. One preferred quantifying method is to use confocal microscope and fluorescent labels. The GeneChip.RTM. system (Affymetrix, Santa Clara, Calif.) is particularly suitable for quantifying the hybridization; however, it will be apparent to those of skill in the art that any similar systems or other effectively equivalent detection methods can also be used.

High density arrays are suitable for quantifying a small variations in expression levels of a gene in the presence of a large population of heterogeneous nucleic acids. Such high density arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. Suitable nucleic acids are also produced by amplification of templates. As a nonlimiting illustration, polymerase chain reaction, and/or in vitro transcription, are suitable nucleic acid amplification methods.

Synthesized oligonucleotide arrays are particularly preferred for this invention. Oligonucleotide arrays have numerous advantages, as opposed to other methods, such as efficiency of production, reduced intra- and inter array variability, increased information content and high signal-to-noise ratio.

(3) Histochemical Techniques

The antibodies of the present invention can be used in a variety of in vitro histochemical techniques for detection of GPR protein. These include enzyme linked immunosorbent assays (ELISAs), Western blots, immunoprecipitations, FACS sorting and immunofluorescence assays common in the art. Alternatively, the protein can also be detected in vivo in a subject by introducing into the subject a labeled anti-GPR 39 antibody. For example, the antibody can be labeled with a radioactive marker whose presence and location in a subject can be detected by standard imaging techniques. Particularly useful are methods which detect the allelic variant of a receptor protein expressed in a subject and methods which detect fragments of a receptor protein in a sample.

Using the above-mentioned techniques, anti-GPR 39 antibodies are useful to detect the presence of GPR 39 protein in cells or tissues to determine the pattern of expression of the receptor among various tissues in an organism and over the course of development. Further, the antibodies can be used to assess receptor expression in disease states, such as in active stages of the disease or in an individual with a predisposition toward disease related to GPR 39 function, such as neoplastic cell formation. When a disorder is caused by an inappropriate tissue distribution, developmental expression, or level of expression of the GPR 39 protein, the antibody can be prepared against the normal receptor protein. If a disorder is characterized by a specific mutation in the GPR 39 protein, antibodies specific for this mutant protein can be used to assay for the presence of the specific mutant GPR 39 protein. However, intracellularly-made antibodies (“intrabodies”) are also encompassed, which would recognize intracellular GPR 39 peptide regions.

The antibodies can also be used to assess normal and aberrant subcellular localization of cells in the various tissues in an organism. Antibodies can be developed against the whole receptor or portions of the receptor, for example, portions of the amino terminal extracellular domain or extracellular loops.

Finally, the antibodies useful as diagnostic tools as an immunological marker for aberrant receptor protein analyzed by electrophoretic mobility, isoelectric point, tryptic peptide digest, and other physical assays known to those in the art. The antibodies are also useful for tissue typing. Thus, where a specific receptor protein has been correlated with expression in a specific tissue, antibodies that are specific for this receptor protein can be used to identify a tissue type.

d) Detecting GPR 39 Activation

(1) Transfluor

Transfluor™ technology is a universal, cell-based, high-content screening assay for known and orphan GPCRs. Transfluor™ technology takes advantage of a mechanism for desensitization of the activated receptor that is common to all GPCRs. The mechanism involves the redistribution of small molecules, termed arresting, from the cytoplasm to the cell membrane in response to GPCR activation. The arrestins bind to the activated receptor, triggering a recycling event that inactivates the GPCR. By fluorescently labeling the arresting, the translocation process can be tracked in real time.

Transfluor™ is quantitated on multiple automated image analysis systems, achieving high signal to background ratios (5:1 to 25:1) and screening rates of 50,000-100,000 compounds per day. The assay discriminates between agonists, partial agonists, and antagonists while providing valuable pharmacological information on efficacy and potency. In contrast to present methods of screening GPCRs, the power of the Transfluor™ assay is in its simplicity, sensitivity, and applicability to all GPCRs without requiring prior knowledge of natural ligands or how a given receptor is coupled to downstream signaling pathways. Transfluor™ kits are available commercially from Norak Biosciences, Inc., 7030 Kit Creek Road, Morrisville, N.C. 27560.

(2) Enzyme and Ion Channel-Linked Assays

G protein coupled receptors (GPCR) are coupled to a variety of heterotrimeric G proteins, which are comprised of α, β, and γ subunits. Upon agonist binding to a GPCR at the cell surface, conformational changes occur within the agonist:GPCR complex which lead to the dissociation of the G protein a subunit from the β αand γ subunits. The G_{α}. and G_{βγ} subunits then stimulate a variety of intracellular effectors, which transduce the extracellular signal to the inside of the cell. Various signal transduction systems known to be coupled to GPCRs include adenylate cyclase, phospholipase C, phospholipase A₂, sodium/hydrogen exchange, calcium mobilization, etc. Thus, measurements of intracellular calcium concentrations and adenylate cyclase activity indicate whether a hit or test compound is functionally behaving as an agonist or antagonist at the neurotensin receptor.

In a specific embodiment, G-protein signal transduction is coupled to expression of a reporter gene, thus permitting a reporter gene screening assay.

Calcium Mobilization Assay

Whole cells expressing the GPR 39 are loaded with a fluorescent dye that chelates calcium ions, such as FURA-2. Upon addition of a compound that binds specifically and modulates GPR 39 activity to these cells, calcium is released from the intracellular stores. The dye chelates these calcium ions. Spectrophotometric determination of the ratio for dye:calcium complexes to free dye determine the changes in intracellular calcium concentrations. Hits from screens of test compounds can be assayed to functionally characterize them as agonists or antagonists. Increases in intracellular calcium concentrations are expected for compounds with agonist activity while compounds with antagonist activity are expected to block stimulated increases in intracellular calcium concentrations.

Cylic AMP Accumulation Assay

Upon agonist binding, Gₛ coupled GPCRs stimulate adenylate cyclase. Adenylate cyclase catalyzes the production of cyclic AMP from adenosine-5′-triphosphate which, in turn, activates protein kinases. G₁ coupled GPCRs are also coupled to adenylate cyclase, however, agonist binding to these receptors results in the inhibition of adenylate cyclase and the subsequent inhibition of cAMP. To measure the inhibition of cAMP accumulation, cells expressing G₁ coupled receptors must first be stimulated to elevate cAMP levels. This is achieved by treating the cells with forskolin, a diterpene that directly stimulates cAMP production. Co-incubation of cells expressing G₁ coupled receptors with forskolin and a functional agonist will result in the inhibition of forskolin-stimulated cAMP accumulation. For a cAMP assay, whole cells stably expressing GPR 39 can be incubated with a test compound, and with forskolin plus a test compound. The cells are then lysed and cAMP levels are measured using the [¹²⁵I]cAMP scintillation proximity assay (SPA). Functional agonists of Gₛ coupled receptors are expected to increase cAMP levels above basal levels whereas functional agonists of G₁ coupled receptors are expected to inhibit the forskolin-stimulated cAMP accumulation.

(3) CART

Cart technology utilizes common recombinant techniques referenced above to render a GPCR constitutively active through mutagenesis. Cells expressing constitutively activated receptors are useful for screening compounds that modulate receptor activation. Such cells can be derived from natural sources or can be created by recombinant means that are well known in the art. For example, see Scheer et al., J. Receptor Signal Transduction Res. 17:57-73 (1997); U.S. Pat. No. 5,750,353.

When a GPCR becomes constitutively active, it binds to a G protein (for example G_{q}, Gₛ, Gᵢ, Gₒ) and stimulates the binding of GTP to the G protein. The G protein then acts as a GTPase and slowly hydrolyzes the GTP to GDP, whereby the receptor, under normal conditions, becomes deactivated. However, constitutively activated receptors continue to exchange GDP to GTP. A non-hydrolyzable analog of GTP, [³⁵S]GTPγS, can be used to monitor enhanced binding to membranes which express constitutively activated receptors. It is reported that [³⁵S]GTPγS can be used to monitor G protein coupling to membranes in the absence and presence of ligand. An example of this monitoring, among other examples well-known and available to those in the art, was reported by Traynor and Nahorski in 1995. Generally, this preferred use of this assay system is for initial screening of candidate compounds because the system is generically applicable to all G protein-coupled receptors regardless of the particular G protein that interacts with the intracellular domain of the receptor.

A constitutively activate orphan GPCR, such as GPR 39, can be used to screen for specific binding compounds using reverse pharmacology approach discussed previously. One approach to differentiating between an inverse agonist, agonist, partial agonist or compounds having no affect on such a receptor, is to use a GPCR Fusion Protein comprising the coding regions of GPR 39 and its companion G protein.

The GPCR Fusion Protein is intended to enhance the efficacy of G protein coupling with the GPCR. The GPCR Fusion Protein is important for screening with a constitutively activated GPCR because such an approach increases the signal that is most preferably utilized in such screening techniques. This is important in facilitating a significant “signal to noise” ratio.

The construction of a GPCR Fusion Protein expression system is within the purview of those having ordinary skill in the art. Commercially available expression vectors and systems offer a variety of approaches that can fit the particular needs of an investigator. Important criteria is that the GPCR sequence and the G protein sequence both be in-frame (preferably, the sequence for the GPCR is upstream of the G protein sequence) to and that the “stop” codon of the GPCR must be deleted or replaced such that upon expression of the GPCR, the G protein can also be expressed. The GPCR can be linked directly to the G protein, or there can be spacer residues between the two (preferably, no more than about 12, although this number can be readily ascertained by one of ordinary skill in the art). Both approaches have been evaluated, and in terms of measurement of the activity of the GPCR, the results are substantially the same; however, there is a preference (based upon convenience) for use of a spacer in that some restriction sites that are not used will, upon expression, effectively, become a spacer. Most preferably, the G protein that couples to the endogenous GPCR will have been identified prior to the creation of the GPCR Fusion Protein construct. Because there are only a few G proteins that have been identified, it is preferred that a construct comprising the sequence of the G protein (i.e., a universal G protein construct) be available for insertion of an endogenous GPCR sequence therein; this provides for efficiency in the context of large-scale screening of a variety of different endogenous GPCRs having different sequences.

e) Detecting Phenotypic Alterations

(1) Soft Agar Growth

Anchorage-independent growth in a soft agar assay is a measure of tumorigenicity that can be used as a selection process to identify compounds that specifically bind and antagonize GPR 39 activity. Non-cancerous parent cells and cancerous cells treated with GPR 39 antagonists that cause reversion from the cancerous phenotype cannot grow in soft agar.

When the combinatorial libraries of the present invention are introduced to the cancerous cells expressing GPR 39, those cells that are cultured in soft agar with library members that specifically bind and/or antagonize GPR 39 die. Conversely, cells that are treated with library members that do not specifically bind and antagonize GPR 39 are able to proliferate under the selection process.

IV. Pharmaceutical Compositions

Compounds found to specifically bind to and modulate GPR 39 can be formulated into pharmaceutical compositions using techniques well known to those in the art. Suitable pharmaceutically-acceptable carriers are available to those in the art; for example, see Remington's Pharmaceutical Sciences, 16^{th} Edition, 1980, Mack Publishing Co., (Oslo et al., eds.).

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a receptor protein or anti-receptor antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al., PNAS 91:3054-3057 (1994)). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

The receptor polynucleotides are useful as a hybridization probe for cDNA and genomic DNA to isolate a full-length cDNA and genomic clones encoding the polypeptide described in SEQ ID NO 1 or SEQ ID NO 4 and to isolate cDNA and genomic clones that correspond to variants producing the same polypeptide shown in SEQ ID NO 1 or SEQ ID NO 4 or the other variants described herein. Variants can be isolated from the same tissue and organism from which the polypeptide shown in SEQ ID NO 1 or SEQ ID NO 4 was isolated, different tissues from the same organism, or from different organisms. This method is useful for isolating genes and cDNA that are developmentally-controlled and therefore may be expressed in the same tissue or different tissues at different points in the development of an organism.

The probe can correspond to any sequence along the entire length of the gene encoding the receptor. Accordingly, it could be derived from 5′ noncoding regions, the coding region, and 3′ noncoding regions. It is understood, however, that the probe would not encompass a fragment already described prior to the invention.

The nucleic acid probe can be, for example, the full-length cDNA of SEQ ID NO 1 or SEQ ID NO 4, or a fragment thereof, such as an oligonucleotide of at least 10, 12, 15, 30, 50, 100, 250 or 500 nucleotides in length and sufficient to specifically hybridize under stringent conditions to mRNA or DNA.

Fragments of the polynucleotides described herein are also useful to synthesize larger fragments or full-length polynucleotides described herein. For example, a fragment can be hybridized to any portion of an mRNA and a larger or full-length cDNA can be produced.

The receptor polynucleotides are also useful as primers for PCR to amplify any given region of a receptor polynucleotide.

The receptor polynucleotides are also useful for constructing recombinant vectors. Such vectors include expression vectors that express a portion of, or all of, the receptor polypeptides. Vectors also include insertion vectors, used to integrate into another polynucleotide sequence, such as into the cellular genome, to alter in situ expression of receptor genes and gene products. For example, an endogenous receptor coding sequence can be replaced via homologous recombination with all or part of the coding region containing one or more specifically introduced mutations.

The receptor polynucleotides are also useful as probes for determining the chromosomal positions of the receptor polynucleotides by means of in situ hybridization methods.

The receptor polynucleotide probes are also useful to determine patterns of the presence of the gene encoding the receptors and their variants with respect to tissue distribution, for example, whether gene duplication has occurred and whether the duplication occurs in all or only a subset of tissues. The genes can be naturally-occurring or can have been introduced into a cell, tissue, or organism exogenously.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

What is claimed is:

1. A method of identifying anticancer agents that modulate GPR 39 protein said method comprising the steps of:
a) contacting GPR 39-specific binding agents to cancer cells; and,
b) detecting anticancer activity to identify an anticancer agent.

2. A method of claim 1 further comprising the step of:
binding a population of different compositions to GPR 39 protein to select GPR 39-specific binding agents.

3. The method of claim 2, wherein the GPR 39 protein has at least 60% homology to SEQ. ID Z.

4. The method of claim 1 wherein the cancer cells further comprise a BRCA-1 sensitive phenotype.

5. A method of inhibiting cancer characteristics in cancer cells by downmodulating GPR 39 protein activity to a level sufficient to inhibit the cancer characteristics of the cancer cells.

6. The method of claim 5 wherein down-modulating comprises contacting the cancer cells with an intrabody.

7. The method of claim 5 wherein down-modulating comprises contacting the cancer cells with an antisense molecule.

8. The method of claim 5 wherein down-modulating comprises contacting the cancer cells with a ribozyme.

9. The method of claim 5 wherein down-modulating comprises contacting the cancer cells with an antagonizing antibody.

10. The method of claim 5 wherein down-modulating comprises contacting the cancer cells with an siRNA.

11. A system for identifying anticancer agents that modulate GPR 39 protein, said system comprising:
a) a container containing GPR 39-specific binding agents; and,
b) a container housing cancer cells that express GPR 39 protein.

12. The system of claim 11, wherein the GPR 39 protein has at least 60% homology to SEQ ID Z.

13. The system of claim 11, wherein the cancer cells further comprise a BRCA-1 sensitive phenotype.

14. The system of claim 11, wherein the cancer cells are selected from the group consisting of: breast, ovarian, prostate, brain, and lung cancer cells.

15. The system of claim 11, wherein the binding agents are antibodies.

16. The system of claim 11, wherein the binding agents are nucleic acids.

17. The system of claim 11, wherein the binding agents are peptides.

18. The system of claim 17, wherein the peptides bind to the transmembrane portion of the GPR 39 protein.

19. A recombinant expression cassette comprising a non-native promoter operably linked to a gene encoding human GPR 39 protein.

20. The recombinant expression cassette of claim 19, wherein the GPR 39 protein has at least 60% homology to SEQ ID NO: 2.

21. An antibody specifically recognizing a peptide having a sequence of at least five amino acids found in a GPR 39 protein having at least 60% homology to SEQ ID NO: 2.

22. The antibody of claim 21, wherein binding of the antibody to the GPR 39 protein down-regulates GPR 39 activity.

23. The antibody of claim 21, wherein binding of the antibody to a GPR 39-expressing cancer cell inhibits the cancer characteristics of the cell.
